(19) United States
(12) Patent Application Publication
(10) Pub. No.: US 2010/0094292 A1
(43) Pub. Date: Apr. 15, 2010

(54) MODULAR INTRAMEDULLARY NAIL

(75) Inventor: Russell M. Parrott, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/576,698

(22) Filed: Oct. 9, 2009

Related U.S. Application Data
(60) Provisional application No. 61/105,069, filed on Oct. 14, 2008.

(51) Int. Cl. A61B 17/58

(52) U.S. Cl. 606/62

(57) ABSTRACT

An intramedullary nail having at least a first segment and a second segment. An exterior surface of the first segment includes a plurality of open spaces therein to permit bone growth into the intramedullary nail. Such bone ingrowth may enhance the fixation between the intramedullary nail and the bone. The first and second segments are detachably secured to one another to accommodate a prosthetic implant.

### CROSS REFERENCE TO RELATED APPLICATION

This application claims priority from U.S. Provisional Patent Application No. 61/105,069, entitled “Modular Intramedullary Nail,” filed on Oct. 14, 2008, by the same inventor hereof, the disclosure of which is expressly incorporated herein by reference.

### BACKGROUND

1. Field of the Invention

The present invention relates to an intramedullary nail. More particularly, the present invention relates to a modular intramedullary nail and to a method for using the same.

2. Description of the Related Art

An intramedullary nail may be used to align and stabilize a fracture of a long bone. For example, in a fractured femur, an intramedullary nail may be inserted into an intramedullary canal of the femur to align and stabilize the fracture. The intramedullary nail may include bores that receive screws or other attachment devices for securing the intramedullary nail to the bone. To stabilize opposing portions of the fractured bone, the intramedullary nail itself or screws extending through the intramedullary nail may extend across the fracture line of the femur. For example, if the head of a long bone, such as the head of a femur, has fractured, screws may extend through bores of the intramedullary nail, across the fracture line, and into the head of the femur.

For various reasons, the intramedullary nail may be removed after the fractured bone heals. For example, the intramedullary nail may be removed if the nail causes the patient pain or discomfort or if the bone becomes infected. Also, the intramedullary nail may be removed if the patient must undergo a subsequent arthroplasty procedure to accommodate a prosthetic implant, such as a prosthetic hip stem. Therefore, known intramedullary nails are constructed of solid, non-porous materials that permit subsequent removal of the intramedullary nail.

### SUMMARY

The present invention provides an intramedullary nail having at least a first segment and a second segment. An exterior surface of the first segment includes a plurality of open spaces therein to permit bone growth into the intramedullary nail. Such bone ingrowth may enhance the fixation between the intramedullary nail and the bone. The first and second segments are detachably secured to one another to accommodate a subsequent prosthetic implant.

According to an embodiment of the present invention, an intramedullary nail is provided that includes an elongate body sized for receipt within a bone and having at least one bore extending therethrough, the at least one bore sized to receive a fixation device for securing the elongate body to the bone. The elongate body includes a first segment and a second segment. The first segment has an exterior surface configured to contact the bone, the exterior surface of the first segment having a plurality of open spaces therein. The second segment is detachably secured to the first segment of the elongate body.

According to another embodiment of the present invention, an intramedullary nail is provided that includes an elongate body sized for receipt within a bone. The elongate body includes a first segment and a second segment. The first segment has an exterior portion configured to contact the bone and an interior portion, the exterior portion of the first segment having a plurality of open spaces therein and the interior portion of the first segment being hollow. The second segment is detachably secured to the first segment of the elongate body.

According to yet another embodiment of the present invention, a method is provided for performing revision surgery. The method includes the steps of providing access to an intramedullary nail implanted in a bone, the intramedullary nail including a first segment and a second segment, the first segment having an exterior surface with a plurality of open spaces therein; separating the second segment of the intramedullary nail from the first segment of the intramedullary nail; and removing the second segment of the intramedullary nail from the bone while retaining the first segment of the intramedullary nail within the bone.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a cross-sectional view of a femur with an orthopedic assembly of the present invention implanted therein;

FIG. 2A is an elevational view of an intramedullary nail of the present invention;

FIG. 2B is an elevational view of another intramedullary nail of the present invention;

FIG. 3A is a cross-sectional view of the intramedullary nail of FIG. 2A, taken along line 3A-3A of FIG. 2A;

FIG. 3B is a cross-sectional view of the intramedullary nail of FIG. 2B, taken along line 3B-3B of FIG. 2B;

FIG. 4A is a cross-sectional view of the intramedullary nail of FIG. 2A, taken along line 4A-4A of FIG. 2A;

FIG. 4B is a cross-sectional view of the intramedullary nail of FIG. 2B, taken along line 4B-4B of FIG. 2B;

FIG. 5A is a cross-sectional view of a femur with a prosthetic hip stem and middle and distal segments of an intramedullary nail implanted therein;

FIG. 5B is a cross-sectional view of a femur with a prosthetic hip stem and middle and distal segments of an intramedullary nail implanted therein; and

FIG. 5C is a cross-sectional view of a femur with a distal femoral prosthetic knee stem and middle and proximal segments of an intramedullary nail implanted therein.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

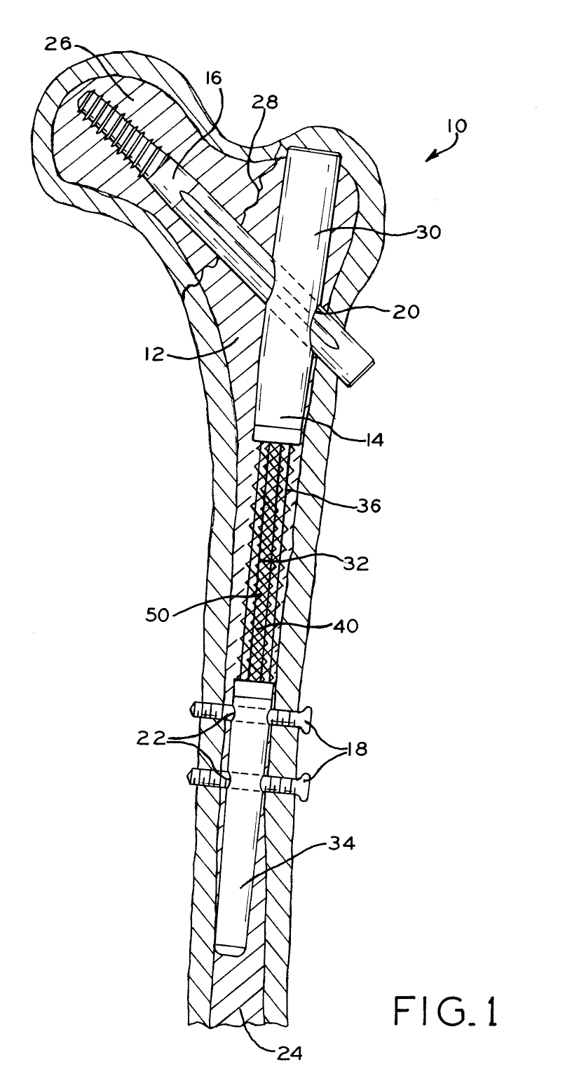

Referring to FIG. 1, orthopedic assembly 10 is shown implanted into femur 12. Although orthopedic assembly 10 is described and depicted herein as being implanted into femur 12, orthopedic assembly 10 may be used in other long bones of the body, such as the tibia, fibula, radius, ulna, clavicle, and other long bones. Orthopedic assembly 10 includes intramedullary nail 14, transverse member or lag screw 16, and distal screws 18. Intramedullary nail 14 of orthopedic assembly 10 includes transverse bore 20 for receiving lag screw 16 and distal bores 22 for receiving distal screws 18.

During surgery, intramedullary nail 14 is implanted into prepared intramedullary canal 24 of femur 12. To prevent rotation and removal of intramedullary nail 14, a surgeon implants distal screws 18 into femur 12 through distal bores 22 of intramedullary nail 14. In cases of a fracture or other trauma to femoral head 26, such as fracture 28 shown in FIG. 1, a surgeon implants lag screw 16 into femoral head 26 of femur 12 through transverse bore 20 of intramedullary nail 14.

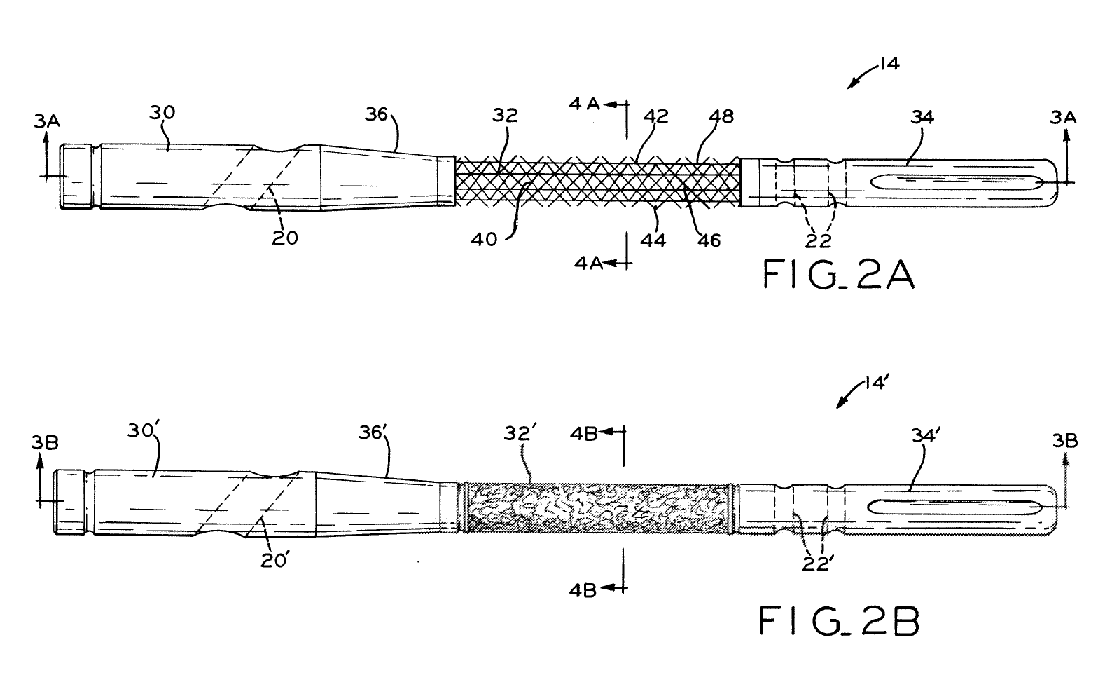

Referring next to FIG. 2A, intramedullary nail 14 of the present invention includes proximal segment 30, middle segment 32, and distal segment 34. Transverse bore 20 for receiving lag screw 16 (FIG. 1) extends through proximal segment 30, and distal bores 22 for receiving distal screws 18 (FIG. 1) extend through distal segment 34.

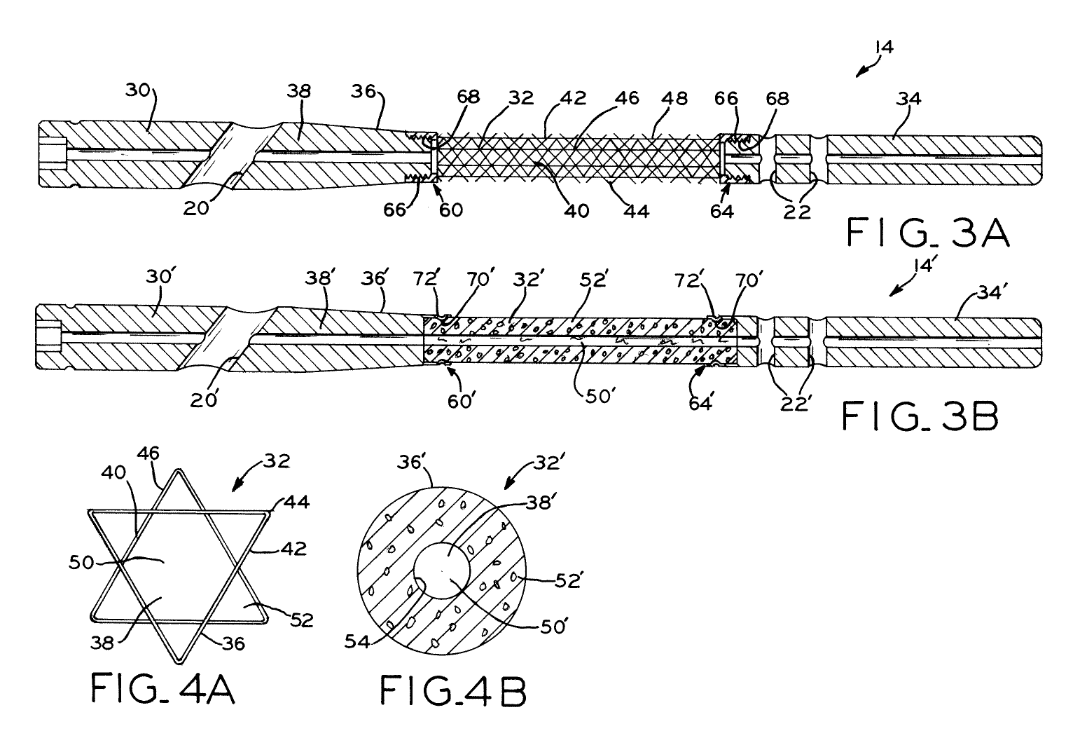

Referring to FIG. 3A, intramedullary nail 14 includes exterior portion 36 and interior portion 38. Exterior portion 36 of intramedullary nail 14 is configured to contact bone of femur 12 (FIG. 1). Interior portion 38 of intramedullary nail 14 is located radially within the surrounding exterior portion 36.

Proximal segment 30 and distal segment 34 of intramedullary nail 14 may be constructed of any suitable biocompatible material. For example, proximal segment 30 and distal segment 34 may be constructed of a biocompatible ceramic, a rigid, biocompatible thermoplastic or fiber reinforced thermoplastic material, including, but not limited to, carbon fiber reinforced poly ether ether ketone (PEEK), or a biocompatible metal, including, but not limited to, titanium, a titanium alloy, cobalt chromium, or cobalt chromium molybdenum. Exterior portion 36 of proximal segment 30 and distal segment 34 of intramedullary nail 14 may be smooth, solid, and non-porous so that a surgeon can later remove proximal segment 30 and/or distal segment 34 from femur 12 (FIG. 1). Interior portion 38 of proximal segment 30 and distal segment 34 of intramedullary nail 14 may be hollow or cannulated and capable of receiving a guide wire, for example.

Middle segment 32 of intramedullary nail 14 may be constructed of a three dimensional truss structure, also known as space truss structure 40. Space truss structure 40 includes support members 42 that extend in three dimensions and are joined together at joints 44 formed at the ends of support members 42. Support members 42 may be straight, rigid objects defining open spaces 52 therebetween.

According to an exemplary embodiment of the present invention, space truss structure 40 may include both diagonal support members 46 and axial support members 48. As shown in FIG. 3A, diagonal support members 46 wind helically around a central axis of space truss structure 40, and axial support members 48 run parallel to the central axis of space truss structure 40 and interconnect with diagonal support members 46 at joints 44.

According to another exemplary embodiment of the present invention, interior portion 38 of space truss structure 40 may be hollow, which portion is referred to herein as channel 50. Channel 50 may be defined along the central axis of space truss structure 40 between support members 42, for example. As shown in FIG. 4A, channel 50 may be defined between support members 42 of space truss structure 40 having a cross-sectional shape of overlapping polygons, such as triangles, squares, hexagons, or octagons.

An exemplary space truss structure 40 is disclosed in U.S. Pat. No. 5,921,048, the disclosure of which is expressly incorporated herein by reference. Also, an exemplary space truss structure 40 is the IsoTruss™ structure generally currently available from Advanced Composite Solutions, LLC, of Payson, Utah. IsoTruss™ is a registered trademark of Brigham Young University of Provo, Utah.

Advantageously, middle segment 32 of intramedullary nail 14 may be constructed of space truss structure 40 to provide durability and rigidity to intramedullary nail 14 when implanted in femur 12 (FIG. 1). Also advantageously, middle segment 32 of intramedullary nail 14 may be constructed of space truss structure 40 to permit bone growth into exterior portion 36 of intramedullary nail 14. Specifically, middle segment 32 of intramedullary nail 14 may permit bone growth into open spaces 52 defined between support members 42 of middle segment 32. Such bone ingrowth into middle segment 32 of intramedullary nail 14 may provide fixation of intramedullary nail 14 to femur 12 (FIG. 1). Middle segment 32 of intramedullary nail 14 may also be provided with osteoconductive materials or osteoinductive materials to enhance bone growth. For example, if channel 50 of middle segment 32 is packed with osteoconductive materials or osteoinductive materials, these materials will travel through open spaces 52 of space truss structure 40 and toward femur 12 to encourage bone growth into middle segment 32.

Referring next to FIG. 2B, another intramedullary nail 14′ of the present invention is provided. Corresponding portions of intramedullary nail 14′ and intramedullary nail 14 are labeled with corresponding reference numerals. Intramedullary nail 14′ includes proximal segment 30′, middle segment 32′, and distal segment 34′. Transverse bore 20′ for receiving lag screw 16 (FIG. 1) extends through proximal segment 30′, and distal bores 22′ for receiving distal screws 18 (FIG. 1) extend through distal segment 34′.

Referring to FIG. 3B, intramedullary nail 14 includes exterior portion 36′ and interior portion 38′. Exterior portion 36′ of intramedullary nail 14′ is configured to contact bone of femur 12 (FIG. 1). Interior portion 38′ of intramedullary nail 14′ is located radially within the surrounding exterior portion 36′.

Proximal segment 30′ and distal segment 34′ of intramedullary nail 14′ may be constructed of any suitable biocompatible material. For example, proximal segment 30′ and distal segment 34′ may be constructed of a biocompatible ceramic, a rigid, biocompatible thermoplastic or fiber reinforced thermoplastic material, including, but not limited to, carbon fiber reinforced poly ether ether ketone (PEEK), or a biocompatible metal, including, but not limited to, titanium, a titanium alloy, cobalt chromium, or cobalt chromium molybdenum. Exterior portion 36′ of proximal segment 30′ and distal segment 34′ of intramedullary nail 14′ may be smooth, solid, and non-porous so that a surgeon can later remove proximal segment 30′ and/or distal segment 34′ from femur 12 (FIG. 1). Interior portion 38′ of proximal segment 30′ and distal segment 34′ of intramedullary nail 14′ may be hollow or cannulated and capable of receiving a guide wire, for example.

Middle segment 32′ of intramedullary nail 14′ may be constructed of a porous material, such as an open-cell material. As used herein, an “open-cell material” is a material containing a plurality of struts defining pores or open spaces 52′ that are connected to each other and form an interconnected network. Middle segment 32′ of intramedullary nail 14′ may have a porosity as low as 55, 60, or 65 percent and as high as 80, 85, or 90 percent or more.

According to an exemplary embodiment of the present invention, interior portion 38′ of middle segment 32′ of intramedullary nail 14′ may be hollow, which portion is referred to herein as channel 50′. Channel 50′ may be defined along the central axis of middle segment 32′. As shown in FIG. 4B, channel 50′ may be defined within hollow rod 54′ that is formed entirely of a porous material. In another embodiment, hollow rod 54′ could include a solid substrate that is coated by a porous material, such as a wire mesh or a beaded material.

An exemplary porous, open-cell material is produced using Trabecular Metal™ technology generally available from Zimmer, Inc., of Warsaw, Ind. Trabecular Metal™ is a trademark of Zimmer Technology, Inc. Such a material may be formed from a reticulated vitreous carbon foam substrate which is infiltrated and coated with a biocompatible metal, such as tantalum, by a chemical vapor deposition (“CVD”) process in the manner disclosed in detail in U.S. Pat. No. 5,282,861, the disclosure of which is expressly incorporated herein by reference. In addition to tantalum, other metals such as niobium, or alloys of tantalum and niobium with one another or with other metals may also be used.

Generally, the porous tantalum structure includes a large plurality of struts defining the open cells, or open spaces, therebetween, with each strut generally including a carbon core covered by a thin film of metal such as tantalum, for example. The open spaces between the struts form a matrix of continuous channels having no dead ends, such that growth of cancellous bone through the porous tantalum structure is uninhibited. The porous tantalum may have a porosity as low as 55, 60, or 65 percent and as high as 80, 85, or 90 percent or more. Thus, porous tantalum is a lightweight, strong porous structure which is substantially uniform and consistent in composition, and closely resembles the structure of natural cancellous bone, thereby providing a matrix into which cancellous bone may grow to provide fixation of intramedullary nail 14′ to femur 12 (FIG. 1).

The porous tantalum structure may be made in a variety of densities to selectively tailor the structure for particular applications. In particular, as discussed in the above-incorporated U.S. Pat. No. 5,282,861, the porous tantalum may be fabricated to virtually any desired porosity and pore size, and can thus be matched with the surrounding natural bone to provide an improved matrix for bone ingrowth and mineralization.

Advantageously, middle segment 32′ of intramedullary nail 14′ may be constructed of a porous material to provide durability and rigidity to intramedullary nail 14′ when implanted in femur 12 (FIG. 1). Also advantageously, middle segment 32′ of intramedullary nail 14′ may be constructed of a porous material to permit bone growth into exterior portion 36′ of intramedullary nail 14′. Specifically, middle segment 32′ of intramedullary nail 14′ may permit bone growth into open spaces 52′, such as the pores defined between the fibrous struts of middle segment 32′. Such bone ingrowth into middle segment 32′ of intramedullary nail 14′ may provide fixation of intramedullary nail 14′ to femur 12 (FIG. 1). Middle segment 32′ of intramedullary nail 14′ may also be provided with osteoconductive materials or osteoinductive materials to enhance bone growth.

Referring again to FIG. 1, an exemplary intramedullary nail 14 of the present invention is configured to permit bone growth into intramedullary nail 14. Such bone growth into intramedullary nail 14, specifically middle segment 32 intramedullary nail 14, may enhance the fixation between intramedullary nail 14 and femur 12. However, a patient may later require a subsequent arthroplasty procedure. For example, if fracture 28 in femur 12 does not heal adequately, a surgeon may choose to perform a partial hip arthroplasty procedure, replacing femoral head 26 of femur 12 with a prosthetic implant.

Middle segment 32 of intramedullary nail 14 may be detachably secured to proximal segment 30 and/or distal segment 34. For example, in the embodiment of FIG. 3A, middle segment 32 includes proximal end 60 and distal end 64 having female threads 66 that are configured to detachably mate with corresponding male threads 68 of proximal segment 30 and distal segment 34, respectively. As another example, in the embodiment of FIG. 3B, middle segment 32′ includes proximal end 60′ and distal end 64′ having annular groove 70′ configured to detachably mate with corresponding annular ridges 72′ of proximal segment 30′ and distal segment 34′, respectively. The segments may also have tapered engagements. It is also within the scope of the present invention that a separate fastener device may be used to detachably secure middle segment 32 of intramedullary nail 14 to proximal segment 30 and distal segment 34. For example, a screw may be driven from proximal segment 30 into middle segment 32. Further, it is within the scope of the present invention that middle segment 32 may be secured to proximal segment 30 differently than middle segment 32 is secured to distal segment 34.

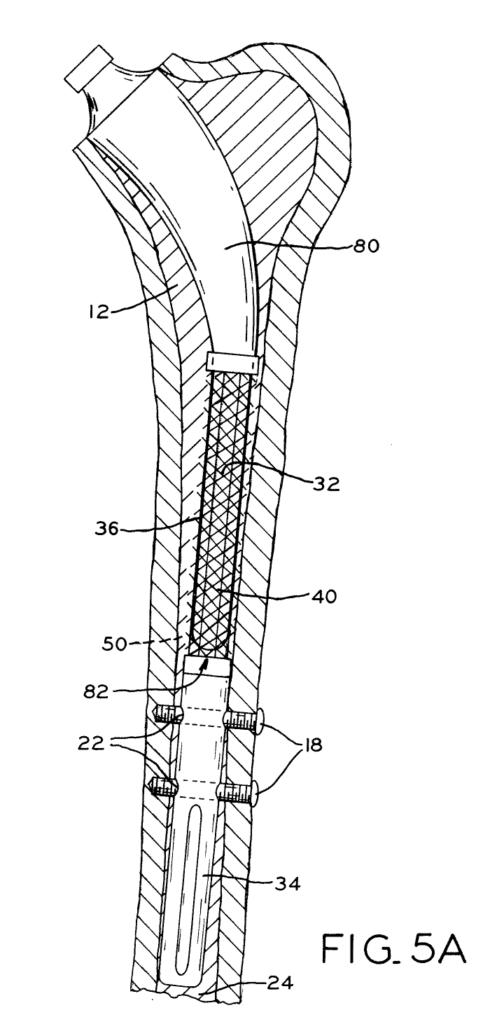

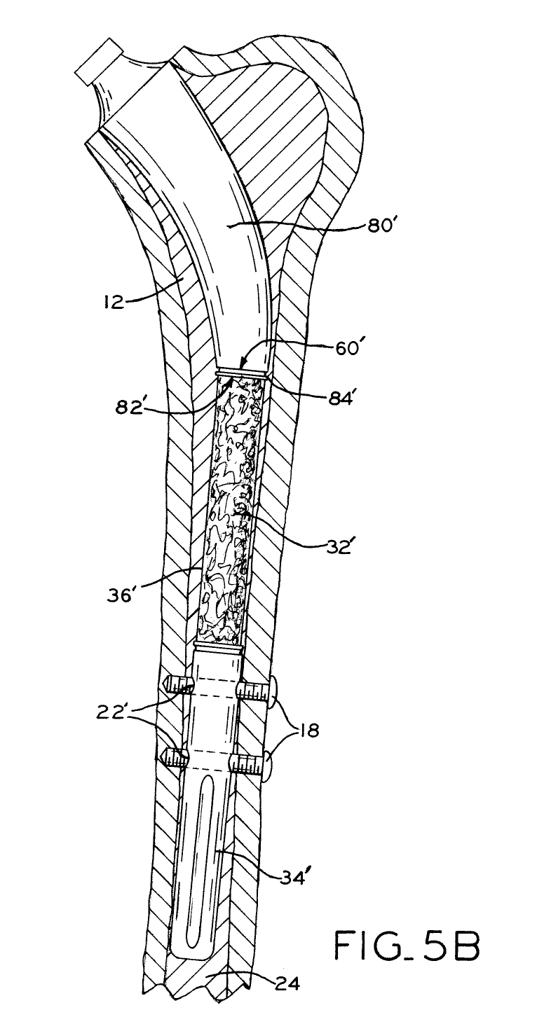

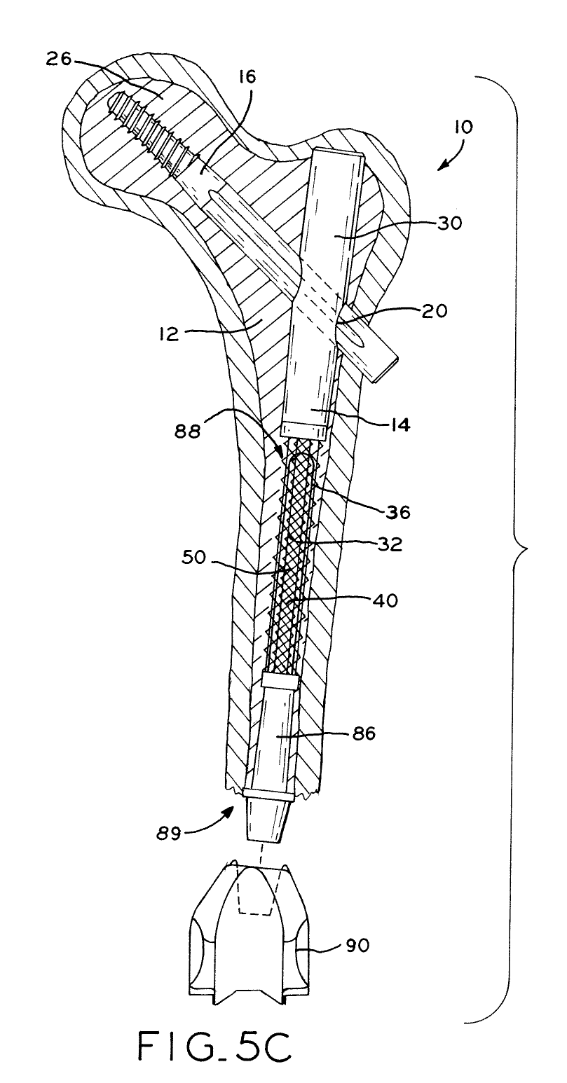

Prior to or during a subsequent arthroplasty procedure, lag screw 16 and/or distal screws 18 (FIG. 1) may be removed from intramedullary nail 14. Next, proximal segment 30 and/or distal segment 34 of intramedullary nail 14 may be separated or detached from middle segment 32 of intramedullary nail 14. Then, proximal segment 30 and/or distal segment 34 of intramedullary nail 14 may be removed from intramedullary canal 24 of femur 12 while middle segment 32 of intramedullary nail 14 is retained within intramedullary canal 24 of femur 12. For example, proximal segment 30 may be removed proximally from intramedullary canal 24 of femur 12, and distal segment 34 may be removed distally from intramedullary canal 24 of femur 12. The ingrowth of bone into middle segment 32 of intramedullary nail 14 may enhance the fixation between the retained middle segment 32 of intramedullary nail 14 and femur 12. Finally, a prosthetic implant, such as proximal femoral hip stem 80 (FIG. 5A), proximal femoral hip stem 80′ (FIG. 5B), or distal femoral knee stem 86 (FIG. 5C), may be coupled to middle segment 32 of intramedullary nail 14.

According to an exemplary embodiment of the present invention, illustrated in FIG. 5A, proximal femoral hip stem 80 may be implanted into femur 12 through middle segment 32 of intramedullary nail 14. Specifically, proximal femoral hip stem 80 may include distal end 82 that extends into channel 50 of middle segment 32 of intramedullary nail 14. As shown in FIG. 5A, proximal segment 30 of intramedullary nail 14 (FIG. 2A) was removed from femur 12 to accommodate proximal femoral hip stem 80. Over the life of proximal femoral hip stem 80, middle segment 32 and the retained distal segment 34, including distal screws 18, of intramedullary nail 14 may enhance the fixation between proximal femoral hip stem 80 and femur 12.

According to another exemplary embodiment of the present invention, illustrated in FIG. 5B, proximal femoral hip stem 80′ may be implanted into femur 12 and attached to middle segment 32′ of intramedullary nail 14′. Specifically, proximal femoral hip stem 80′ may include distal end 82′ that attaches to middle segment 32′ of intramedullary nail 14′, and more specifically to proximal end 60′ of middle segment 32′ of intramedullary nail 14′. Distal end 82′ of proximal femoral hip stem 80′ may attach to middle segment 32′ of intramedullary nail 14′ in the same manner that proximal segment 30′ (FIG. 3B) once attached to middle segment 32′ of intramedullary nail 14′. For example, if proximal end 60′ of middle segment 32′ includes annular groove 70′ (FIG. 3B), distal end 82′ of proximal femoral hip stem 80′ may include annular ridge 84′ configured to mate with groove 70′ of middle segment 32′. As shown in FIG. 5B, proximal segment 30′ of intramedullary nail 14′ (FIG. 3B) was removed from femur 12 to accommodate proximal femoral hip stem 80′. Over the life of proximal femoral hip stem 80′, middle segment 32′ and the retained distal segment 34′, including distal screws 18, may enhance the fixation between proximal femoral hip stem 80′ and femur 12.

According to yet another exemplary embodiment of the present invention, illustrated in FIG. 5C, distal segment 34 of intramedullary nail 14 may be removed from femur 12 (FIG. 1) to accommodate distal femoral knee stem 86, for example. Over the life of distal femoral knee stem 86, middle segment 32 and the retained proximal segment 30 of intramedullary nail 14 may enhance the fixation between distal femoral knee stem 86 and femur 12. Distal femoral knee stem 86 may include proximal end 88 that extends into channel 50 of middle segment 32, as shown in FIG. 5C, or distal femoral knee stem 86 may attach to distal end 64′ of middle segment 32′ (as shown in FIG. 5B with respect to proximal femoral hip stem 80′). Also, distal femoral knee stem 86 may include distal end 89 that is configured to couple to an articulating femoral component 90.

To accommodate either proximal femoral hip stem 80 (FIG. 5A) or proximal femoral hip stem 80′ (FIG. 5B) in the proximal end of femur 12 and distal femoral knee stem 86 (FIG. 5C) in the distal end of femur 12, both proximal segment 30 and distal segment 34 of intramedullary nail 14 may be removed from intramedullary canal 24 of femur 12 (FIG. 1). Middle segment 32 of intramedullary nail 14 may be retained within intramedullary canal 24 of femur 12 and coupled to both proximal femoral hip stem 80, 80′, and distal femoral knee stem 86.

Advantageously, intramedullary nail 14 may enhance the fixation between intramedullary nail 14 and femur 12 while also accommodating a subsequent prosthetic implant. The above-described intramedullary nail 14 may be modified to accommodate various bones, fractures, and prosthetic implants. For example, the number and arrangement of the modular segments of intramedullary nail 14 may vary.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An intramedullary nail comprising:
an elongate body sized for receipt within a bone and having at least one bore extending therethrough, the at least one bore sized to receive a fixation device for securing the elongate body to the bone, the elongate body comprising:
a first segment having an exterior surface configured to contact the bone, the exterior surface of the first segment having a plurality of open spaces therein; and
a second segment detachably secured to the first segment of the elongate body.

2. The intramedullary nail of claim 1, wherein the first segment of the elongate body comprises a hollow interior sized to receive an implant stem therethrough.

3. The intramedullary nail of claim 2, wherein the plurality of open spaces extend through the first segment from the exterior surface to the hollow interior.

4. The intramedullary nail of claim 1, wherein the first segment of the elongate body includes an end configured to mate with a corresponding end of an implant stem.

5. The intramedullary nail of claim 1, further comprising a third segment of the elongate body, wherein the first segment of the elongate body includes a proximal end configured to detachably mate with the second segment of the elongate body and a distal end configured to detachably mate with the third segment of the elongate body.

6. The intramedullary nail of claim 5, wherein the second segment of the elongate body comprises the at least one bore sized to receive the fixation device in the form of a proximal screw, and wherein the third segment of the elongate body comprises at least one distal bore extending therethrough that is sized to receive a distal screw.

7. The intramedullary nail of claim 1, wherein the first segment of the elongate body comprises a space truss structure having a hollow interior.

8. The intramedullary nail of claim 1, wherein the first segment of the elongate body comprises a space truss structure having a plurality of support members defining open spaces therebetween.

9. The intramedullary nail of claim 1, wherein the first segment of the elongate body comprises a space truss structure having a cross-sectional shape comprising a plurality of overlapping polygons.

10. The intramedullary nail of claim 1, wherein the first segment of the elongate body comprises a porous material having a plurality of pores, whereby the plurality of open spaces in the first segment comprise the plurality of pores.

11. An intramedullary nail comprising:
an elongate body sized for receipt within a bone, the elongate body comprising:
a first segment having an exterior portion configured to contact the bone and an interior portion, the exterior portion of the first segment having a plurality of open spaces therein and the interior portion of the first segment being hollow; and
a second segment detachably secured to the first segment of the elongate body.

12. The intramedullary nail of claim 11, wherein the hollow interior portion of the first segment of the elongate body is sized to receive an implant stem.

13. The intramedullary nail of claim 11, wherein the first segment of the elongate body includes an end configured to mate with a corresponding end of an implant stem.

14. The intramedullary nail of claim 11, further comprising a third segment of the elongate body, wherein the first segment of the elongate body includes a proximal end configured to detachably mate with the second segment of the elongate body and a distal end configured to detachably mate with the third segment of the elongate body.

15. The intramedullary nail of claim 14, wherein the second segment of the elongate body comprises a proximal bore extending therethrough that is sized to receive a proximal screw, and wherein the third segment of the elongate body comprises at least one distal bore extending therethrough that is sized to receive a distal screw.

16. The intramedullary nail of claim 11, wherein the first segment of the elongate body comprises a space truss structure having a plurality of support members, whereby the plurality of open spaces in the first segment of the elongate body are defined between the plurality of support members.

17. The intramedullary nail of claim 11, wherein the first segment of the elongate body comprises a space truss structure having a cross-sectional shape comprising a plurality of overlapping polygons.

18. The intramedullary nail of claim 11, wherein the first segment of the elongate body comprises a hollow rod coated with a porous material having a plurality of pores, whereby the plurality of open spaces in the first segment comprise the plurality of pores.

19. A method of performing revision surgery comprising the steps of:
providing access to an intramedullary nail implanted in a bone, the intramedullary nail comprising a first segment and a second segment, the first segment having an exterior surface with a plurality of open spaces therein;
separating the second segment of the intramedullary nail from the first segment of the intramedullary nail; and
removing the second segment of the intramedullary nail from the bone while retaining the first segment of the intramedullary nail within the bone.

20. The method of claim 19, further comprising the steps of:
implanting a prosthetic implant into the bone; and
coupling the prosthetic implant to the retained first segment of the intramedullary nail.

21. The method of claim 20, wherein the step of coupling the prosthetic implant to the first segment of the intramedullary nail comprises inserting a stem of the prosthetic implant into a hollow interior of the first segment.

22. The method of claim 20, wherein the step of coupling the prosthetic implant to the first segment of the intramedullary nail comprises attaching an end of the prosthetic implant to a corresponding end of the first segment.

23. The method of claim 20, wherein the prosthetic implant comprises one of a proximal femoral hip stem and a distal femoral knee stem.
